(19) United States
(12) Patent Application Publication
(10) Pub. No.: US 2006/0135643 A1
(43) Pub. Date: Jun. 22, 2006

(54) Laser curable polymerisable composition for the protection of hard tissue

(75) Inventors: Joachim E. Klee, Radolfzell (DE); Andreas Facher, Konstanz (DE); Christoph Weber, Konstanz (DE); Rolf Mulhaupt, Freiburg (DE); Martin Schmider, Freiburg (DE)

Correspondence Address: Douglas J. Hura, Esquire;DENTSPLY International Inc., 570 West College Avenue, P.O. Box 872, York, PA, 17405-0872, US

(73) Assignee: DENTSPLY De Trey GmbH, Konstanz (DE)

(21) Appl. No.: 11/217,570

(22) Filed: Sep. 1, 2005

Related U.S. Application Data
(60) Provisional application No. 60/606,510, filed on Sep. 2, 2004.

(30) Foreign Application Priority Data
Mar. 11, 2004 (EP) 04 005 802.6

(51) Int. Cl. A61K 6/08

(52) U.S. Cl. 523/116

(57) ABSTRACT

One-component heat-curable sealant composition having a viscosity of at most 5 Pas (23° C.) for the protection of exposed dental surfaces, comprising (a) a polymerisable monomer and/or oligomer having at least two polymerizable double bonds per molecule, and (b) an initiator system comprising a thermally stable initiator having a 10 hour half-life decomposition temperature of from 95° C. to 135° (0.1M in benzene); wherein the composition further contains a precursor for a filler which is an alkoxysilane compound undergoing polycondensation reactions during heat curing of the composition, thereby forming a filler.

### RELATED APPLICATIONS

This application is a U.S. Ordinary application, which claims the benefit from both U.S. Provisional Application No. 60/606,510 filed Sep. 2, 2004 and EP Application No. 04 005 802.6 filed Mar. 11, 2004.

### FIELD OF THE INVENTION

The present invention relates to a one-component heat-curable sealant composition having a viscosity of at most 5 Pas (23° C.) for the protection of hard tissue, in particular exposed dental surfaces. The present invention also relates to a process for the protection of hard tissue, in particular exposed dental surfaces. The sealant composition is characterized by a superior shelf-life and by being capable of undergoing a rapid rate of cure by a laser without heat-damaging neighboring tissue. The sealant composition of the invention has improved biocompatibility, high hardness, low long-term abrasion and high acid resistance.

### TECHNICAL BACKGROUND

Thermosetting dental materials are known from U.S. Pat. No. 4,866,146. A polymerizable dental composition containing 2,5-dimethyl-2,5-di(benzoylperoxy)hexane (DHPBZ), tert.-butylperoxy-3,3,5-trimethylhexanoate (TBPIN), tert.-butylperoxy benzoate (TBPB), tert.-butylamyl peroxide, or di(tert.-butyl)peroxide (DTBP) is known from EP-A 0 951 894. A method for curing a dental composition by using a laser is known from U.S. Pat. No. 6,168,431.

Protective dental treatment for avoiding mechanical, bacterial or chemical trauma, in particular primary and secondary caries, is of increasing importance in the dental field. Pit and fissure sealants are known as compositions used in protective treatments of occlusal surfaces. Sealant materials for protective treatment of cervical surfaces exposed by gingival retraction caused by long-standing periodontitis are also known. However, such materials are characterized by high abrasion and require frequent replacements.

Dental materials can be divided into chemically (thermally) curable materials and materials polymerizing by exposure to light. Thermal polymerization is usually severely limited for applications on living tissue or other heat sensitive surfaces. Highly reactive initiators and the presence of amine accelerators are usually required whereby the shelf-life of a one-component composition is deteriorated or multi-component systems are required.

Pit and fissure sealants are typically based on methacrylate monomers. Self-curing compositions are typically two-component systems including in a first component one or more methacrylate monomers and at least one component of a free radical liberating (redox) polymerization system for said monomer(s). The monomer composition may include the peroxy type catalyst (oxidant) which is later contacted with a second component including the reducing agent (reductant) shortly prior to dental use. In case of a sealant composition, the viscosity of the composition must be low enough to allow thorough penetration of fissures and intricate interdental spaces with no air bubbles prior to polymerization. The handling of a two-component system for providing a low-viscosity composition is highly problematic.

Light-curing compositions contain methacrylate monomers and an initiator system in a single pack. However, the storage stability of such compositions depends on the absence of light and the careful handling of the composition prior to polymerization.

A primary object of the invention is to provide polymerizable dental sealant compositions wherein the foregoing and related disadvantages are eliminated or at least mitigated to a substantial extent.

Another object of the invention is to provide polymerizable dental sealant compositions capable of undergoing a rapid rate of cure to produce a polymerizate having strong adhesion to dentin or enamel and having excellent protective properties as a pit or fissure sealant or a sealant for exposed cervical surfaces.

Yet another object of the invention is to provide such a sealant composition having good structural stability within the environment of the human oral cavity. Still another object of the invention is to provide such a composition wherein any requirements for using higher catalyst concentrations to achieve effective rate and degree of cure are obviated. Still another object of the invention is to provide a sealant composition having a high shelf-stability even in the presence of an amine accelerator.

Yet a still further object of the invention is to provide a process of utilizing such compositions to prepare a high quality polymerizate.

### DISCLOSURE OF THE INVENTION

The present invention provides a one-component heat-curable sealant composition having a dynamic viscosity of at most 5 Pas (23° C.) for the protection of exposed dental surfaces, comprising
(a) a polymerisable monomer and/or oligomer having at least two polymerizable double bonds per molecule, and
(b) an initiator system comprising a thermally stable initiator having a 10 hour half-life decomposition temperature of from 95° C. to 135° C.
   wherein the composition further contains a precursor for a filler which is an alkoxysilane compound undergoing polycondensation reactions during heat curing of the composition, thereby forming a filler.

The polymerizable monomers or oligomers in the one-component heat-curable sealant composition according to the invention are capable of free-radical polymerization and are preferably (meth)acrylate monomers or oligomers. The (meth)acrylate monomer or oligomer is selected from materials having at least two, and preferably two to four polymerizable double bonds per molecule so that the cured sealant composition be crosslinked and thus better suited for use in the oral cavity. Monomers with a single polymerisable double-bond may be used in order to adjust the viscosity of the composition. (Meth)acrylate monomer materials useful herein are well known in the art. The preferred materials generally include monomers having a central portion containing an organic moiety and at least two (meth)acrylic end groups. Preferable monomers are ethyleneglycol dimethacrylate, diethyleneglycol dimethacrylate, dodecanediol dimethacrylate, trimethylolpropane tri(meth) acrylate, hydroxyethyl methacrylate, triethyleneglycol dimethacrylate, trimethylolpropane triacrylate, glycerine dimethacrylate, and methacrylic acid. Desirable characteristics for such monomers and/or oligomers include good film forming properties, low viscosity, low polymerization shrinkage, low water sorption and the ability to cure rapidly and completely in the mouth when irradiated with a laser. It is also desirable that the monomers be low in volatility and non-irritating to the tooth pulp. An example of preferred oligomers is a condensation product of methacryloyloxypropyl oxycarbonylamido propyltriethoxy silane. A mixture of two or more appropriate methacrylate monomers is within the scope of this invention. In fact, depending on the choice of monomers, mixture are often highly desirable to optimize the characteristics of the resulting dental composition.

Thus, it is preferred that the monomer or oligomer or monomer or oligomer blend has a viscosity of at most 5 Pas at 23° C.

The initiator system comprises a thermally stable initiator having a 10 hour half-life decomposition temperature of from 95° C. to 135° C. The 10 hour half-life decomposition temperature is measured based on a 0.1M solution of the initiator in a suitable hydrocarbon solvent such as preferably benzene, or alternatively toluene, isododecane, mineral oil, or styrene. The thermal polymerization system may be selected from the group of peroxides, peroxide/amine redox systems and azobis compounds.

Peroxy catalysts useful herein and capable of initiating polymerization of the methacrylate monomer(s) include, without limitation,

tert.-butyl peroxybenzoate,

cumene hydroperoxide,

a combination of tert.-butyl peroxy benzoate/N.N′-diethylamino-p-benzoic acid ethyl ester, as well as other conventional peroxy compounds such as

| | |
|---|---|
| 1,1-di(tert.-butylperoxy)3,3,5-trimethyl cyclohexane (TMCH), | T_{1/2}^{10 h} = 95° C. (0.1 M in isodecane); |
| 1,1-di(tert.-butylperoxy) cyclohexane (CH), | T_{1/2}^{10 h} = 97° C. (0.1 M in isodecane); |
| tert.-butylperoxy isopropyl carbonate, | T_{1/2}^{10 h} = 97° C. (0.1 M in benzene); |
| tert.-butylperoxy-3,3,5-trimethylhexanoate (TBPIN) | T_{1/2}^{10 h} = 100° C. (0.1 M in benzene); |
| 2,5-dimethyl-2,5-di(benzoylperoxy)hexane (DHPBZ) | T_{1/2}^{10 h} = 100° C. (0.1 M in benzene); |
| tert.-butylperoxy(2-ethylhexyl)carbonate (TBP EHC) | T_{1/2}^{10 h} = 100° C. (0.1 M in isodecane); |
| tert.-butylperoxy acetate | T_{1/2}^{10 h} = 102° C. (0.1 M in benzene) |
| tert.-amylperoxy benzoate (TABP) | T_{1/2}^{10 h} = 102° C. (0.1 M in isodecane) |
| tert.-butylperoxy benzoate (TBPB) | T_{1/2}^{10 h} = 104° C. (0.1 M in benzene) |
| 2,2-di(tert.-butylperoxy)butan)(BU) | T_{1/2}^{10 h} = 104° C. (0.1 M in isodecane) |
| n-butyl-4,4-di(tert.-butylperoxy)valerate (NBV) | T_{1/2}^{10 h} = 110° C. (0.1 M in benzene) |
| ethyl-3,3-di(tert.-butylperoxy)butyrate (EBU) | T_{1/2}^{10 h} = 114° C. (0.1 M in mineral oil) |
| dicumyl peroxide (DCUP) | T_{1/2}^{10 h} = 116° C. (0.1 M in benzene) |
| tert.-butylcumyl peroxide (BCUP) | T_{1/2}^{10 h} = 118° C. (0.1 M in benzene) |
| di-(tert.-amyl)peroxide (DTAB) | T_{1/2}^{10 h} = 118° C. (0.1 M in styrene) |
| di(2-tert.-butylperoxyisopropyl)benzene (DIPP) | T_{1/2}^{10 h} = 120° C. (0.1 M in benzene) |
| 2,5-dimethyl-2,5-di(tert.-butylperoxy)hexane (DHBP) | T_{1/2}^{10 h} = 120° C. (0.1 M in benzene) |
| di(tert.-butyl)peroxide (DTBP) | T_{1/2}^{10 h} = 125° C. (0.1 M in benzene) |
| 2,5-dimethyl-2,5-di(tert.-butylperoxy)hexyne-3 (DYBP) | T_{1/2}^{10 h} = 128° C. (0.1 M in benzene) |
| 3,3,6,6,9,9,-hexamethyl-1,2,4,5-tetraoxacyclononane (HMCN) | T_{1/2}^{10 h} = 135° C. (0.1 M in isodecane). |

If desired, peroxide stabilizers such as ascorbic acid, maleic acid and the like may be included in small amounts.

The initiator system of the one-component heat-curable sealant composition according to the invention may further comprises an amine. The amines in the one-component heat-curable sealant composition according to the invention are preferably substituted or unsubstituted aliphatic, alkyl, aryl, or cycloalkyl primary amines, secondary amines, primary-secondary amines, primary-tertiary amines or secondary-tertiary amines.

The azobis initiator may be

| | |
|---|---|
| 2-(carbamoylazo)isobutyronitrile | T_{1/2}^{10 h} = 104° C. (0.1 M in toluene) |
| 2,2′-azobis(2,4,4-trimethylpentane) | T_{1/2}^{10 h} = 110° C. (0.1 M in |
| | diphenylether). |

The one-component heat-curable sealant composition contains a precursor for a filler. The precursor for a filler is one or more alkoxysilane compounds undergoing polycondensation reactions during heat curing of the composition, thereby forming the filler. The alkoxysilane compound is a silicon compound characterized by one or more hydrolysable alkoxy groups. The alkoxysilane compound may be a compound of the following formula:
RₙSi(OR′₄₋ₙ),
wherein n is 0, 1 or 2; R, which are independent from each other are selected from alkyl, aryl or an organofuctional moiety, and R′, which are independent from each other, are selected from a straight chain or branched C₁ to C₈ alkyl group. Preferably, an organofunctional moiety contains a polymerizable double bond which may take part in a chain growth polymerization of the a polymerisable monomer and/or oligomer of component (a). The alkoxy groups OR′ may be selected from groups such as methoxy, ethoxy, propoxy, n-butoxy, i-butoxy, t-butoxy. In one embodiment, the alkoxysilane compound may be a compound of the formula Si(OR′₄) wherein R′ is as defined above. As an example, tetraethoxysilane (TEOS) may be mentioned. Alternatively, the alkoxysilane compound may be a compound of the formula RSi(OR′)₃, wherein n is 1, and R and R′ are as defined above. As an example, methacryloyloxypropyl oxycarbonylamido propyltriethoxy silane may be mentioned. Moreover, the alkoxysilane compound may be an oligomeric siloxane compound obtainable by partial condensation of one or more of the above alkoxysilane compounds. As an example, a condensation product of methacryloyloxypropyloxycarbonylamidopropyltriethoxy silane may be mentioned. The precursor for a filler undergoes polycondensation reactions during heat curing of the composition, thereby forming filler. The polycondensation is facilitated by traces of moisture present under application conditions which allows the formation of silanols, and the high temperature generated locally when the composition is cured.

In a further embodiment of the present invention, the one-component heat-curable sealant composition may further comprise (c) a heat-curable step-growth polymerization system. Preferably, the heat-curable step-growth polymerization system is selected from the group consisting of epoxide-amine, epoxide-thiol, epoxide-carboxylic acid, epoxide-carboxylic acid anhydride, epoxide-phenol, isocyanate-amine, isocyanate-alcohol, isocyanate-thiol, isothiocyanate-amine, isothiocyanate-alcohol, isothiocyanate-thiol, carboxylic acid derivative-amine, carboxylic acid derivative-alcohol, carboxylic acid derivative-thiol, acrylate-amine, acrylate-thiol, acrylamide-amine, acrylamide-thiol, maleinimide-amine, maleinimide-thiol, acrylate-malonic acid derivative, acrylamide-malonic acid derivative, blocked isocyanate-amine, blocked isocyanate-alcohol, SiH-En addition, and siloxane systems. The SiH-en addition system may be a silane-acrylate, silane-allylether, silane-vinylether, silane-acrylamide, or silane-maleinimide system. The most preferred heat-curable step-growth polymerization system is a siloxane system.

The one-component heat-curable sealant composition may further comprise (d) a filler. The filler may be an inorganic filler or an organic filler or a mixture thereof. The inorganic particulate filler employed in the compositions of this invention include fused silica, quartz, crystaline silica, amorphous silica, soda glass beads, glass rods, ceramic oxides, particulate silicate glass, radiopaque glasses (barium and strontium glasses), and synthetic minerals. It is also possible to employ finely divided materials and powdered hydroxylapatite, although materials that react with silane coupling agents are preferred. Also available as a filler are colloidal or submicron silicas coated with a polymer. Small amounts of pigments to allow matching of the composition to various shades of teeth can be included. The filler particles would be generally smaller than about 5 microns in diameter and preferably smaller than 3 μm, preferably in a range of from 3 to 500 nm. The filler in one-component heat-curable sealant composition according to the invention preferably comprises fine teflon particles.

In a further preferred embodiment of the present invention, the filler comprises a nanofiller, particularly modified silica according to the following formula:

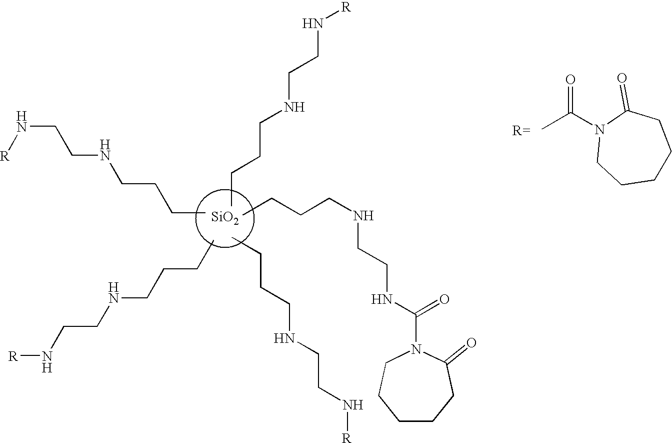

The one-component heat-curable sealant composition may further comprise (e) a solvent. Suitable solvents are selected from organic solvents such as ethanol, tert.-butanol and acetone.

The one-component heat-curable sealant composition according to the invention is polymerisable by locally heating the composition at a temperature of between 120 and 250° C., preferably between 160 to 220° C. without damage of hard tissue whereby a dental/medical coating is obtained.

The one-component heat-curable sealant composition according to the invention preferably is a pit and fissure sealant or a cervical surface sealant.

The one-component heat-curable sealant composition according to the invention preferably contains the polymerisable monomer and/or oligomer in an amount of from 10 to 99 wt.-%.

The one-component heat-curable sealant composition according to the invention preferably contains the thermally stable initiator in an amount of from 0.01 to 5.0 wt.-%, preferably in an amount of from 0.1 to 2 wt-%.

The one-component heat-curable sealant composition according to the invention preferably contains the precursor of a filler in an amount of from 5 to 70 wt.-%, preferably from 10 to 60 wt.-%.

The one-component heat-curable sealant composition according to the invention preferably contains the filler in an amount of from 0 to 30 wt.-%.

The one-component heat-curable sealant composition according to the invention preferably contains the solvent in an amount of from 0 to 70 wt.-%.

In a preferred embodiment, the one-component heat-curable sealant composition according to the invention consists essentially of
10 to 99 wt.-% polymerisable monomer and/or oligome having at least two polymerizable double bonds per molecule;
0.01 to 5.0 wt.-% initiator having a 10 hour half-life decomposition temperature of from 95° C. to 135° (0.1M in benzene);
10 to 60 wt.-% alkoxysilane compound undergoing polycondensation reactions during heat curing of the composition, thereby forming a filler,
0 to 30 wt.-% filler, and
0 to 70 wt.-% solvent.

The present invention further provides a process for the protection of exposed dental surfaces or heat sensitive dental products, which comprises the following steps:
(a) applying a one-component heat-curable sealant composition having a viscosity of at most 5 Pas (23° C.), which comprises
   (a1) a polymerisable monomer and/or oligomer having at least two polymerizable double bonds per molecule, and
   (a2) an initiator system comprising a thermally stable initiator having a 10 hour half-life decomposition temperature of from 95° C. to 135° (0.1M in benzene); to an exposed surface of a tooth for providing a coating on the exposed surface of the tooth, and
(b) heating the coating obtained in step (a) to a temperature of at least 100° C. for curing the coating and forming a protective sealant coating.

In a preferred embodiment, the one-component heat-curable sealant composition further contains a precursor for a filler which is an alkoxysilane compound undergoing polycondensation reactions during heat curing of the composition, thereby forming a filler.

The heating in the process according to the intention may be performed especially by irradiation laser light, infrared light, or microwaves, preferably by irradiation of laser light.

Preferably, a protective sealant coating is formed on pits or fissures of a tooth by the process according to the invention. The protective sealant coating may also be formed on a cervical surface of a tooth.

The present invention further relates to a kit-of-parts comprising a one-component heat-curable sealant composition according to a the invention and a laser.

The present invention further relates to the se of a composition according to the invention for the protection of a tooth.

The following examples and comparative examples are illustrative of embodiments of the invention. All parts and percentages are by weight.

### EXAMPLE 1

6.045 g resin matrix composed of dodecanediol dimethacrylate and a condensation product of methacryloyloxypropyl oxycarbonylamido propyltriethoxy silane, 4.030 tetraethoxy silane, 0.155 g tert. butyl peroxy benzoate and 0.103 g dimethylamino benzoic acid ethylester were homogeneously mixed.

In order to dry dentin surface the Laser was applied in a pulse sequence of 10×4 pulses (Fᵤₚ=0.5 J/cm²; fᵤₚ=100 Hz; f=1 Hz) Thereafter, approximately 10 μl of a mixture prepared above were applied homogeneously on dentin that prior was etched for 45 s by using of 37% H₃PO₄. Subsequently, this layer was irradiated by the following pulses: 5×, 10×4 pulses (Fᵤₚ=0.9 J/cm²; fᵤₚ=100 Hz; f=1 Hz) and 1× and 5×4 pulses (Fᵤₚ=1.2 J/cm²; fᵤₚ=100 Hz; f=1 Hz).

The formed layer has a thickness of approximately 7 μm. It withstands a wipe test and scratch test.

### EXAMPLE 2

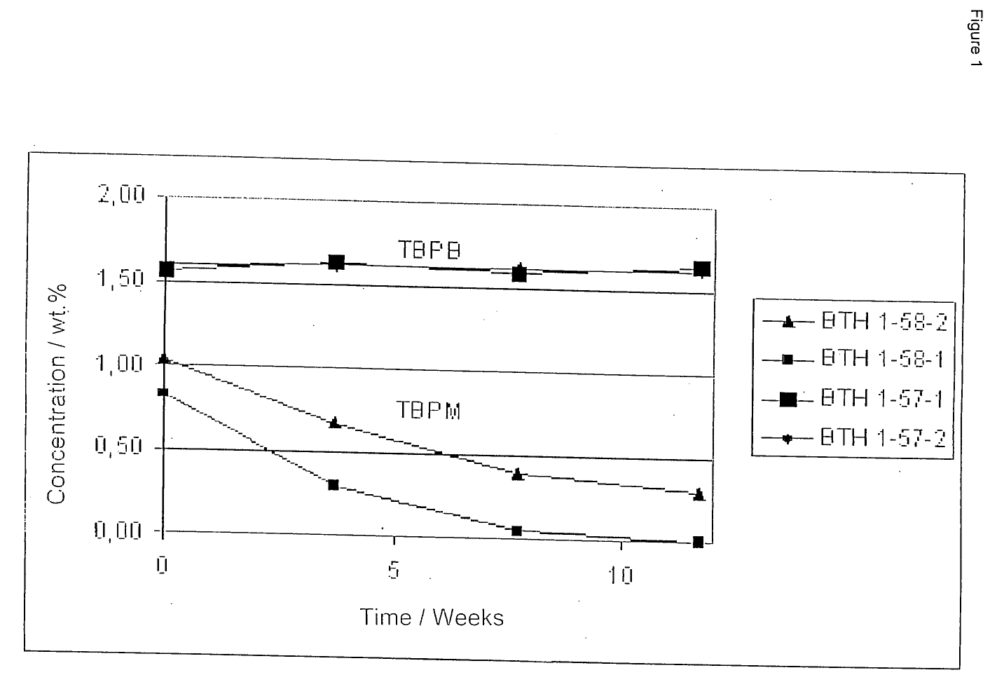

The storage stability of different compositions at a temperature of 23% was investigated. For this purpose, compositions containing initiator as indicated in table 1 were incorporated in a mixture of ORMOSIL Matrix and TEOS (60/40) and stored in closed containers for the time indicated. Analysis was performed in order to determine the remaining amount of catalyst. The results are shown in FIG. 1.

TABLE 1

| | TBPB/% | TBPM/% | DMABE |
|---|---|---|---|
| Example 2-1 | — | 15 | — |
| (Comparative) | | | |
| (BTH 1-58-2) | | | |
| Example 2-2 | — | 15 | 10 |
| (Comparative) | | | |
| (BTH 1-58-1) | | | |
| Example 2-3 | 15 | — | 10 |
| (BTH 1-57-1) | | | |
| Example 2-4 | 15 | — | — |
| (BTH 1-57-2) | | | |

TBPB: tert. butyl peroxy benzoate
TBPM: tert. butyl monoperoxy maleate T_{1/2}^{10 h} = 82° C.(0.1 M in benzene);
DMABE: dimethylaminobenzoic acid ethylester.

As shown by FIG. 1, a composition according to the invention appears to have a superior storage stability even in the presence of an amine accelerator whereas a composition containing an initiator having a 10 hour half-life decomposition temperature of less than 95° C., shows an inferior storage stability.

### COMPARATIVE EXAMPLE 1

In order to dry dentin surface the Laser was applied in a pulse sequence of 10×4 pulses (Fᵤₚ=0.5 J/cm²; fᵤₚ=100 Hz; f=1 Hz) Thereafter, Seal & Protect (Dentsply De Trey) was applied homogeneously on dentin that prior was etched for 45 s by using of 37% age H₃PO₄. Then this layer was irradiated by the following pulses: 5×, 10×4 pulses (Fᵤₚ=0.9 J/cm²; fᵤₚ=100 Hz; f=1 Hz) and 1× and 5×4 pulses (Fᵤₚ=1.2 J/cm²; fᵤₚ=100 Hz; f=1 Hz).

The formed layer has a thickness of approximately 10 μm. It does not withstand a wipe and a scratch test.

1. One-component heat-curable sealant composition having a viscosity of at most 5 Pas (23° C.) for the protection of exposed dental surfaces, comprising
(a) a polymerisable monomer and/or oligomer having at least two polymerizable double bonds per molecule, and
(b) an initiator system comprising a thermally stable initiator having a 10 hour half-life decomposition temperature of from 95° C. to 135° (0.1M in benzene);
wherein the composition further contains a precursor for a filler which is an alkoxysilane compound undergoing polycondensation reactions during heat curing of the composition, thereby forming a filler.

2. The one-component heat-curable sealant composition according to claim 1 further comprising
(c) a heat-curable step-growth polymerization system.

3. The one-component heat-curable sealant composition according to claim 1 further comprising
(d) a filler.

4. The one-component heat-curable sealant composition according to claim 3, further comprising
(e) a solvent.

5. The one-component heat-curable sealant composition according to claim 1, wherein the initiator system further comprises an amine.

6. The one-component heat-curable sealant composition as in claim 1, wherein the initiator system is selected from the group of peroxides, peroxide/amine redox systems and azobis compounds.

7. The one-component heat-curable sealant composition according to claim 6, wherein the initiator system is selected from tert.-butyl peroxybenzoate, cumene hydroperoxide, tert.-butyl peroxy benzoate/N.N′-diethyl-p-benzoic acid ethyl ester, 2-(carbamoylazo)isobutyronitrile, and 2,2′-azobis(2,4,4-trimethylpentane).

8. The one-component heat-curable sealant composition according to claim 1, wherein the polymerizable monomers or oligomers are (meth)acrylate monomers or oligomers.

9. The one-component heat-curable sealant composition according to claim 1, which is polymerisable by locally heating the composition at a temperature of between 120 and 250° C., preferably between 160 to 220° C.

10. The one-component heat-curable sealant composition according to claim 5, wherein the amines are substituted or unsubstituted aliphatic, alkyl, aryl, or cycloalkyl primary amines, secondary amines, primary-secondary amines, primary-tertiary amines or secondary-tertiary amines.

11. The one-component heat-curable sealant composition according to claim 3, wherein the filler comprises fine teflon particles.

12. The one-component heat-curable sealant composition according to claim 1, which is a pit and fissure sealant or a cervical surface sealant.

13. The one-component heat-curable sealant composition according to claim 8, wherein the composition contains the polymerisable monomer and/or oligomer in an amount of from 10 to 99 wt.-%.

14. The one-component heat-curable sealant composition according to claim 1, wherein the composition contains the thermally stable initiator in an amount of from 0.01 to 5.0 wt.-%.

15. The one-component heat-curable sealant composition according to claim 3, wherein the composition contains the filler or precursor thereof in an amount of from 0 to 30 wt.-%.

16. The one-component heat-curable sealant composition according to claim 4, wherein the composition contains the solvent in an amount of from 0 to 70 wt.-%.

17. A process for the protection of exposed dental surfaces or heat sensitive dental products, which comprises the following steps:
(a) applying a one-component heat-curable sealant composition having a viscosity of at most 5 Pas (23° C.), which comprises
(a1) a polymerisable monomer and/or oligomer having at least two polymerizable double bonds per molecule, and
(a2) an initiator system comprising a thermally stable initiator having a 10 hour half-life decomposition temperature of from 95° C. to 135° (0.1M in benzene); to an exposed surface of a tooth for providing a coating on the exposed surface of the tooth, and
(b) heating the coating obtained in step (a) to a temperature of at least 100° C. for curing the coating and forming a protective sealant coating.

18. The process according to claim 17, wherein the heating is performed by irradiation of laser light.

19. The process according to claim 17, wherein the protective sealant coating is formed on pits or fissures of a tooth.

20. The process according to claim 17, wherein the protective sealant coating is formed on a cervical surface of a tooth.

21. Kit-of-parts comprising a one-component heat-curable sealant composition according to claim 1 and a laser.
